SEQUENCE LISTING (2 sequences)

SEQ ID NO: 1 - protein, 16 aa, Artificial Sequence
  Linker Peptide
SPGISGGGGGILDSMG

SEQ ID NO: 2 - protein, 295 aa, Salmonella enterica
AQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNSPGISGGGGGILDSMGTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

(19) United States
(12) Patent Application Publication
(10) Pub. No.: US 2022/0389065 A1
(43) Pub. Date: Dec. 8, 2022

(54) ANTICANCER COMPOSITION COMPRISING TLR5 AGONIST DERIVED FROM FLAGELLIN AS ACTIVE INGREDIENT

(71) Applicant: THE CATHOLIC UNIVERSITY OF KOREA INDUSTRY-ACADEMIC COOPERATION FOUNDATION, Seoul (KR)

(72) Inventors: Seok-Goo CHO, Seoul (KR); Keon-IL IM, Seoul (KR); Nayoun KIM, Seoul (KR); Young-Woo JEON, Seoul (KR); Yunejin SONG, Gyeongsangnam-do (KR); Junseok LEE, Seoul (KR)

(21) Appl. No.: 17/789,300

(22) PCT Filed: May 7, 2021
(86) PCT No.: PCT/KR2021/005734

§ 371 Date: Jun. 27, 2022

(30) Foreign Application Priority Data
Jun. 4, 2020 (KR) 10-2020-0067728

(51) Int. Cl. C07K 14/195; A61K 38/16; C07K 16/28

(52) U.S. Cl. CPC C07K 14/195; A61K 38/164; C07K 16/2818; A61K 2039/505

(57) ABSTRACT

The present invention relates to an anticancer drug composition comprising, as an active ingredient, TLR5 agonist derived from flagellin. The TLR5 agonist derived from flagellin of the present invention can exhibit an anticancer or anticancer adjuvant effect alone or concurrently with an immune checkpoint blockade, and thus, can be developed as a cancer cell growth inhibitory active ingredient.

### TECHNICAL FIELD

The present invention relates to an anticancer composition including a TLR5 agonist derived from flagellin as an active ingredient.

The national R&D project that supported the present invention has the project identification number of 1465029638, the task number of HI14C3417020019, the department name of Ministry of Health and Welfare, the project management organization name of Korean Health Industry Development Institute, the research project name of innovative specialized research project (R&D), the research project name of development of new drugs for the treatment of immune and non-immune tissue damage based on small molecule free radical scavengers, the research period of Aug. 1, 2019 to Nov. 30, 2019.

In addition, the national R&D project that supported the present invention has the task identification number of 1345331440, the task number of 2020R1I1A1A01067669, the department name of Ministry of Education, the project management organization name of National Research Foundation of Korea, the research project name of Science and Engineering Research Foundation (R&D), the research project name of a study on acceleration of immune reconstruction and improvement of residual tumor removal in the transplantation of an autologous hematopoietic stem cell using toll-like receptor ligand in relapsed/refractory lymphoma, the research period of Jun. 1, 2020 to May 31, 2023.

### BACKGROUND ART

Toll-like receptor 5 (TLR5) is a protein encoded by the TLR5 gene in humans (PNAS. 95 (2): 588-93), and is a member of the toll-like receptor (TLR) family. TLR5 is known to be able to recognize flagellin from invading motile bacteria (Seminars in Immunopathology. 29 (3): 275-88). TLR5 is known to be involved in the onset of various diseases, including inflammatory bowel disease (Journal of Physiology and Pharmacology. 60 Suppl 4: 71-5).

Flagellin is a major structural protein constituting bacterial flagellar filaments which are mobile cell organelles Tens of thousands of flagellin molecules are polymerized helically to form long whip-like flagellar filaments. Flagellin includes a D0 domain, a D1 domain, a D2 domain, and a D3 domain. Among them, the D0 domain and the D1 domain are required for filament assembly. The D0 and D1 domains of flagellin are highly conserved in structure and sequence among a variety of bacterial species and function as common molecular patterns of bacteria having flagella to alert the host of bacterial infection. It is known that flagellin is recognized by TLR5 and activates the NF-κB signaling mechanism to induce innate immune stimulation, cytoprotection and radioresistance.

On the other hand, currently, cancer is one of the diseases that account for the largest proportion among the causes of human death. Cancer is a disease caused by changes in normal cells due to mutations in genes caused by various causes, and refers to malignant tumors that do not follow normal cell differentiation, proliferation, and growth patterns. Cancer is characterized by “uncontrolled cell growth”, and by this abnormal cell growth, a cell mass called a tumor is formed and penetrates into surrounding tissues, and in severe cases, it metastasizes to other organs in the body. Cancer is an intractable chronic disease that incurs pain and ultimately death without being able to cure fundamentally in many cases even when treated with surgery, radiation, and drug therapy.

Drug treatment of cancer, that is, anticancer drug, are generally cytotoxic compounds that attack and kill cancer cells, and have high side effects because they damage not only cancer cells but also normal cells. Therefore, targeted anticancer drugs have been developed to reduce such side effects. However, in the case of these targeted anticancer drugs, side effects could be lowered, but there was a limitation in that resistance occurred with a high probability. Therefore, in recent years, interest in immune anticancer drugs that use the body's immune system to reduce the problems due to toxicity and resistance is rapidly increasing. As an example of such an immune anticancer agent, an immune checkpoint blockade that binds to PD-L1 on the surface of cancer cells and inhibits the binding of T cells to PD-1 to activate T cells and attack cancer cells has been developed.

With the recent development of immune checkpoint blockades targeting PD-1 (programmed death-1), CTLA-4 (cytotoxic T-lymphocyte antigen-) and the like, the treatment response rate of tumor patients has been significantly improved. The actual administration of anti-PD-1 showed the tumor response rate of 40 to 45% in melanoma and non-small cell lung cancer, the tumor response rate of 13 to 24% in urothelial cell carcinoma, and the response rate of 87% and the complete remission of 17% in relapsed/refractory Hodgkin's lymphoma. However, the number of carcinomas patients to which the drug is applied is limited, and even if a primary reaction occurs, a problem of rapid recurrence appears.

Accordingly, the present inventors have developed a novel peptide agonist of TLR5 (Toll-like receptor 5) derived from flagellin, and have completed the present invention by experimentally confirming the possibility of using the TLR5 agonist alone or concurrently as an anticancer composition.

### DISCLOSURE

### Technical Problem

An object of the present invention is to provide an anticancer composition including a TLR5 agonist derived from flagellin and a pharmaceutically acceptable carrier.

Other objects and technical features of the present invention are presented in more detail by the following description of the invention, claims and drawings.

### Technical Solution

An embodiment of the present invention relates to an anticancer composition including a therapeutically effective amount of a TLR5 agonist derived from flagellin.

In the present invention, the TLR5 agonist derived from flagellin may include the D0 domain and D1 domain of the flagellin.

In the present invention, the TLR5 agonist derived from flagellin may include a linker peptide of an amino acid sequence set forth in SEQ ID NO: 1 inside the D1 domain.

In the present invention, the TLR5 agonist derived from flagellin may include an amino acid sequence set forth in SEQ ID NO: 2.

In the present invention, the TLR5 agonist derived from flagellin may regulate tumor microenvironment.

In the present invention, the TLR5 agonist derived from flagellin may increase M1 (F4/80+CD206−) polarization and decrease M2 (F4/80+CD206+) polarization in a spleen and lymph node.

In the present invention, the composition may have a growth inhibitory activity of a cancer cell.

In the present invention, the anticancer composition including a therapeutically effective amount of a TLR5 agonist derived from flagellin may further include an immune checkpoint blockade.

In the present invention, the immune checkpoint blockade may be an anti-PD-1 antibody.

In the present invention, the anti-PD-1 antibody may be one or more selected from the group consisting of avelumab, tremelimumab, ipilimumab, nivolumab, pembrolizumav, atezolizumab, durvalumab, lamvrolizumab, AMP-224, MEDI4376 and CT-011.

In the present invention, the anticancer composition including a therapeutically effective amount of a TLR5 agonist derived from flagellin may include a pharmaceutically acceptable carrier.

Another embodiment of the present invention relates to a pharmaceutical agent including an anticancer composition including the therapeutically effective amount of a TLR5 agonist derived from flagellin of the present invention.

### Advantageous Effects

The present invention relates to an anticancer composition including a TLR5 agonist derived from flagellin as an active ingredient. Since the TLR5 agonist derived from flagellin of the present invention can show anticancer or anticancer adjuvant effects either alone or concurrently with immune checkpoint blockade, it can be developed as an active ingredient for inhibiting the growth of cancer cells.

### DESCRIPTION OF DRAWINGS

FIG. 1 is a schematic diagram showing an overall experiment according to the administration of an TLR5 agonist of the present invention, which is a polypeptide of the amino acid sequence of SEQ ID NO: 2 (hereinafter, the TLR5 agonist of SEQ ID NO: 2 also referred to as “KMRC011”), and anti-PD-1 antibody to a C57BL/6 (H-2kb) colorectal cancer mouse animal model transplanted with a MC-38 cell line.

FIG. 2 is a graph of experimental results showing a tumor size for each administered substance after a date of tumor transplantation according to an alone or concurrent administration of a TLR5 agonist of the present invention and an anti-PD-1 antibody in a colorectal cancer mouse model.

FIG. 3 is a photograph of experimental results showing an anticancer inhibitory effect according to an alone or concurrent administration of a TLR5 agonist of the present invention and an anti-PD-1 antibody in a colorectal cancer mouse model.

FIG. 4 is an analysis result of immune profiling of flow cytometry in spleen and lymph node cells according to an alone or concurrent administration of a TLR5 agonist of the present invention and an anti-PD-1 antibody in a colorectal cancer mouse model.

FIG. 5 shows changes in M1 polarization and M2 polarization in spleen and lymph node cells according to an alone or concurrent administration of a TLR5 agonist of the present invention and an anti-PD-1 antibody in a colorectal cancer mouse model.

FIG. 6 is a schematic diagram showing an overall experiment according to an administration of a TLR5 agonist of the present invention, which is a polypeptide of the amino acid sequence of SEQ ID NO: 2, and an anti-PD-1 antibody to a B-cell lymphoma mouse model.

FIG. 7 shows a result of confirming a degree of bioluminescence according to the number of cells by measuring the bioluminescence of different A20-Luc-GFP cell numbers.

FIG. 8 is a graph of experimental results showing a tumor size for each administered substance after a date of tumor transplantation according to an alone or concurrent administration of a TLR5 agonist of the present invention and an anti-PD-1 antibody in a B-cell lymphoma mouse model.

FIG. 9 is a graph of experimental results showing a tumor size for each administered substance after a date of tumor transplantation according to an alone or concurrent administration of a TLR5 agonist of the present invention and an anti-PD-1 antibody in a B-cell lymphoma mouse model.

### BEST MODE

Hereinafter, preferred embodiments of the present invention will be described. However, the embodiment of the present invention may be modified in various other forms, and the scope of the present invention is not limited to the embodiments described below. In addition, the embodiments of the present invention are provided in order to more completely explain the present invention to those of ordinary skill in the art.

In order to achieve the above object, the present invention provides an anticancer composition including a therapeutically effective amount of a TLR5 agonist derived from flagellin.

As used herein, the term “TLR5 agonist derived from flagellin” is meant to include all proteins or polypeptides derived from bacterial flagellin protein or obtained by modifying the same, which have an activity of activating Toll-like receptor 5 (TLR5)-mediated signaling.

As used herein, the term “flagellin” refers to a major protein constituting bacterial flagellar filaments. Flagellin includes a D0 domain, a D1 domain, a D2 domain, and a D3 domain. It is known that flagellin is recognized by TLR5 and activates the NF-κB signaling mechanism to induce innate immune stimulation, cytoprotection and radioresistance.

According to one embodiment of the present invention, the “TLR5 agonist derived from flagellin” may be a peptide substance including the D0 domain of flagellin and the D1 domain of flagellin.

According to another embodiment of the present invention, the “TLR5 agonist derived from flagellin” may include the D0 domain of flagellin, the D1 domain of flagellin, and a linker peptide.

According to still another embodiment of the present invention, the linker peptide may be included within the D1 domain.

According to yet another embodiment of the present invention, the linker peptide may include the amino acid sequence of SEQ ID NO: 1.

According to still yet another embodiment of the present invention, the “TLR5 agonist derived from flagellin” includes the amino acid sequence of SEQ ID NO 2.

According to still yet another embodiment of the present invention, the “TLR5 agonist derived from flagellin” may show the growth inhibitory activity of cancer cells by regulating tumor microenvironment. Specifically, “regulating tumor microenvironment” means increasing M1 (F4/80+CD206−) polarization and decreasing M2 (F4/80+CD206+) polarization in the spleen and lymph nodes.

As used herein, the term “cancer” is a physiological condition in mammals that is usually characterized by uncontrolled cell growth, and refers to all new cell growth and proliferation (whether malignant or benign), and all precancerous and cancerous cells and tissues.

The cancer may be selected from the group consisting of melanoma, skin cancer, lung cancer, liver cancer, gastric cancer, pancreatic cancer, bone cancer, head or neck cancer, uterine cancer, ovarian cancer, breast cancer, fallopian tube carcinoma, endometrial carcinoma, cervical carcinoma, vaginal carcinoma, vulvar carcinoma, Hawkins' disease, esophageal cancer, small intestine cancer, colorectal cancer, colon cancer, rectal cancer, perianal cancer, endocrine gland cancer, thyroid cancer, parathyroid cancer, adrenal cancer, soft tissue sarcoma, prostate cancer, chronic or acute leukemia, lymphocyte lymphoma, bladder cancer, kidney or ureter cancer, renal cell carcinoma, renal pelvic carcinoma, central nervous system tumor, primary central nervous system lymphoma, spinal cord tumor, brainstem glioma and pituitary adenoma, but is not limited thereto. According to one embodiment of the present invention, the TLR5 agonist derived from flagellin of the present invention has a preventive or therapeutic effect on a colorectal cancer. The colorectal cancer refers to a malignant tumor composed of cancer cells generated in the colon.

According to one embodiment of the present invention, the TLR5 agonist derived from flagellin of the present invention has a prophylactic or therapeutic effect on B-cell lymphoma. The B cell lymphoma may be selected from the group consisting of low/follicular non-Hodgkin's lymphoma (NHL), small lymphocytic (SL) NHL, intermediate/follicular NHL, intermediate diffuse NHL, high immunoblast NHL, high lymphoblast NHL, high small non-division cell NHL, bulky disease NHL, and Waldenstrom's macroglobulinemia, but is not limited thereto.

Meanwhile, the composition of the present invention may be provided in the form of an anticancer composition including a therapeutically effective amount of TLR5 agonist derived from flagellin and additionally immune checkpoint blockade.

The immune system of a living body has an immune checkpoint system to suppress the hyperimmune response caused by the overproliferation of T-cells, and the immune checkpoint functions to suppress the hyperimmune response caused by the overactivation and/or hyperproliferation of T-cells. However, cancer cells exploit the immune checkpoint to prevent T-cells from attacking themselves, thereby evading attack by the immune system, thereby causing cancer.

The immune checkpoint blockade includes an antibody that targets an immune checkpoint protein, which is a protein involved in immune checkpoint, and can treat diseases such as cancer, and the immune checkpoint blockade may be an antibody, a fusion protein, an aptamer, or an immune checkpoint protein-binding fragment thereof.

The immune checkpoint blockade may be an anti-immune checkpoint protein antibody or an antigen-binding fragment thereof. Preferably, the immune checkpoint blockade may be selected from an anti-CTLA4 antibody, a derivative thereof or an antigen-binding fragment thereof, anti-PD-1 antibody, a derivative thereof or an antigen-binding fragment thereof; an anti-LAG-3 antibody, a derivative thereof or an antigen-binding fragment thereof; an anti-OX40 antibody, a derivative thereof or an antigen-binding fragment thereof; an anti-TIM3 antibody, a derivative thereof or an antigen-binding fragment thereof; and an anti-PD-1 antibody, a derivative thereof, or an antigen-binding fragment thereof. More preferably, the immune checkpoint blockade may be one or more selected from the group consisting of avelumab, tremelimumab, ipilimumab, nivolumab, pembrolizumav, atezolizumab, durvalumab, lamvrolizumab, AMP-224, MEDI4376 and CT-011, but is not limited thereto.

The composition of the present invention may be provided as an anticancer composition including a therapeutically effective amount of a TLR5 agonist derived from flagellin, and a pharmaceutically acceptable carrier.

As used herein, the term “therapeutically effective amount” refers to an amount suitable for generating an anticancer effect on carcinoma by administering the “TLR5 agonist derived from flagellin” as an active ingredient, to a subject patient. Specifically, the term “therapeutically effective amount” refers to a sufficient amount of an agent or compound being administered which will relieve to some extent one or more of the symptoms of the disease or condition being treated.

The preferred dosage of the anticancer composition including a therapeutically effective amount of a TLR5 agonist derived from flagellin and a pharmaceutically acceptable carrier may be suitably adjusted. Preferably, the daily dosage of the composition may be 50 to 100 μg/kg.

The composition of the present invention may include a pharmaceutically acceptable carrier, in addition to the “TLR5 agonist derived from flagellin” as an active ingredient. Examples of this carrier include, but are not limited to, lactose, dextrose, sucrose, sorbitol, mannitol, starch, gum acacia, calcium phosphate, alginate, gelatin, calcium silicate, microcrystalline cellulose, polyvinyl pyrrolidone, cellulose, water, syrup, methyl cellulose, methylhydroxybenzoate, propylhydroxybenzoate, talc, magnesium stearate, menthol and mineral oil, which are commonly used in formulation.

The composition of the present invention may further include a lubricant, a wetting agent, a sweetening agent, a flavoring agent, an emulsifying agent, a suspending agent, a preservative, and the like, in addition to the above-described ingredients. Suitable pharmaceutically acceptable carriers and agents are described in detail in Remington's Pharmaceutical Sciences (19th ed., 1995).

A suitable dosage of the composition of the present invention may vary depending on factors such as formulation method, administration mode, the patient's age, weight, sex, disease condition, diet, administration time, administration route, excretion rate, and response sensitivity.

The composition of the present invention may be administered orally or parenterally. When the composition is administered parenterally, it may be administered by intravenous injection, subcutaneous injection, intramuscular injection, intraperitoneal injection, transdermal administration, and the like. The concentration of the active ingredient included in the composition of the present invention may be determined in consideration of the purpose of treatment, the condition of the patient, the required period, and the like, and is not limited to a concentration within a specific range.

The composition of the present invention may be prepared in a unit dosage form by formulation using a pharmaceutically acceptable carrier and/or excipient according to a method that may be easily performed by a person of ordinary skill in the art to which the present invention pertains, or may be prepared by introducing it in a multiple-dose container. At this time, the formulation may be in the form of a solution, suspension or emulsion in oil or aqueous medium, or may be in the form of an extract, powder, granules, a tablet or a capsule, and may additionally include a dispersant or stabilizer.

In order to achieve another object of the present invention, the present invention provides a pharmaceutical agent including an anticancer composition including a therapeutically effective amount of TLR5 agonist derived from flagellin and a pharmaceutically acceptable carrier.

In order to achieve the above object, the composition of the present invention may be formulated into a conventional agent in the pharmaceutical field by itself or by being mixed with a carrier commonly acceptable in the pharmaceutical field. Preferably, the conventional formulation may be various formulations such as oral administration formulations such as tablets, capsules, solutions, suspensions, etc., injection formulations or suspensions. In order to prevent the drug from being decomposed by gastric acid during oral administration, the composition may be administered by using an antacid concurrently or by formulating a solid preparation for oral administration, such as a tablet, coated with an enteric coating.

### EXAMPLES

### Experimental Methods

1. Colorectal Cancer Mouse Model

(1) Tumor Mouse Model Establishment and Tumor Size Analysis

The colorectal cancer cell line, MC-38, was purchased from the Korea Cell Line Bank (Seoul, Korea), and was cultured in DMEM medium (Gibco, Carlsbad, Calif., USA) containing 1% antibiotics (10 U/mL penicillin and 10 g/mL streptomycin; Gibco) and 10% heat-inactivated fetal bovine serum (FBS; Gibco) to grow the MC-38 cells.

The grown MC-38 cell line (1×10⁶) was resuspended in sterile physiological saline and implanted subcutaneously into C57BL6 mouse at a dose of 200 μl to introduce a tumor mouse model. In order to compare the anticancer effect of the TLR5 agonist of the present invention, anti-PD-1 was used as a positive control and physiological saline was used as a negative control. In addition, by concurrently administering the anti-PD-1 and TLR5 agonists, the effect of the concurrent administration was also confirmed. The size of the formed tumor was measured at intervals of 2 to 3 days from the eleventh day after transplantation, and was calculated according to the formula of wide²×length×0.5. On days 10 and 26 after transplantation, the mouse was euthanized and spleens and lymph nodes were collected.

(2) Isolation of Tissue Single Cell

The spleen and lymph node tissues collected from the mouse were placed between the rough sides of two opaque slide glasses and the slide glasses were rubbed against each other to separate the tissues into single cell units, and the separated spleen cells were treated with an ammonium-chloride-potassium lysis solution (ACK Lysing Buffer; Gibco) to lyse the red blood cells. The spleen and lymph node cells were then resuspended in RPMI 1640 medium containing 1% antibiotics (10 U/mL penicillin and 10 g/mL streptomycin; Gibco) and 5% heat-inactivated fetal bovine serum (FBS; Gibco).

(3) Flow Cytometry

Mouse's spleen or lymph node cells were evaluated by flow cytometry. To investigate M1 or M2 polarization of macrophages, the spleen and lymph node cells of mouse transplanted with MC-38 cell line were washed with staining buffer and resuspended in staining buffer, followed by immunostaining with anti-mouse CD206 PE (BioLegend, San Diego, Calif., USA) and anti-mouse F4/80 Alexa Fluor 700 (BioLegend, San Diego, Calif., USA) at 4° C. for 30 minutes. After staining, spleen and lymph node cells were washed with staining buffer and resuspended in staining buffer. They were then evaluated using flow cytometry analysis in a FACS_LSR Fortessa (BD Pharmingen, San Diego, Calif., USA) using FlowJo software (TreeStar, Ashland, Oreg., USA).

2. B Cell Lymphoma Mouse Model

(1) Tumor Cell Lines

A20-Luc-GFP, which was a B cell lymphoma cell line, was purchased from Imanis Life Sciences (New York, USA). A20-Luc-GFP is one in which LV-eGFP-P2A-Neo transgene is introduced in the A20 cell line, and expresses luciferase and green fluorescent protein. Luciferase is an enzyme that produces light by oxidizing luciferin, a chemical compound found in the cells of bioluminescent organisms, under the catalytic effect of ATP.

The A20-Luc-GFP cell line was grown in RPMI medium (Gibco, Carlsbad, Calif., USA) containing 1% antibiotics (10 U/mL penicillin and 10 g/mL streptomycin; Gibco) and 10% heat-inactivated fetal bovine serum (FBS; Gibco).

(2) Tumor Mouse Model Establishment and Tumor Size Analysis

The grown A20-Luc-GFP cell line (1×10⁶) was resuspended in sterile physiological saline and implanted subcutaneously in BALB/C mouse at a volume of 200 μl to introduce a tumor mouse model. In order to compare the anticancer effect of the TLR5 agonist of the present invention, anti-PD-1 was used as a positive control and physiological saline was used as a negative control. In addition, by concurrently administering the anti-PD-1 and the TLR5 agonists, the effect of the concurrent administration was also confirmed. From the ninth day after transplantation, the above drug was administered intraperitoneally three times at three-day intervals. The size of the formed tumor was measured every 3 to 4 days from the twelfth day after transplantation.

(3) Bioluminescence Analysis

The A20-Luc-GFP cell line suspended in RPMI medium was dispensed in a 96-well plate at a volume of 200 μl from 1×10² cells to 1×10⁵ cells, luciferin was injected at a concentration of 150 ug/ml, and bioluminescence was measured to determine the degree of bioluminescence according to the number of cells. The luciferin was intraperitoneally injected into tumor-implanted mouse at 150 mg/kg, and the bioluminescence of the luciferase-expressing tumor was measured to determine the degree of tumor growth in vivo. Bioluminescence was measured using an in vivo fluorescence spectrometer (IVIS Lumina XRMS).

To observe the anticancer effect of the TLR5 agonist of the present invention administered alone or concurrently, the growth of the tumor for each administered substance was compared after induction of the tumor mouse model.

### Experimental Results

1. Colorectal Cancer Mouse Model

(1) Verification of Anticancer Effect of the TLR5 Agonist in a Tumor Mouse Model

To observe the anticancer effect of the TLR5 agonist of the present invention administered alone or concurrently, the growth of the tumor for each administered substance was compared after induction of the tumor mouse model.

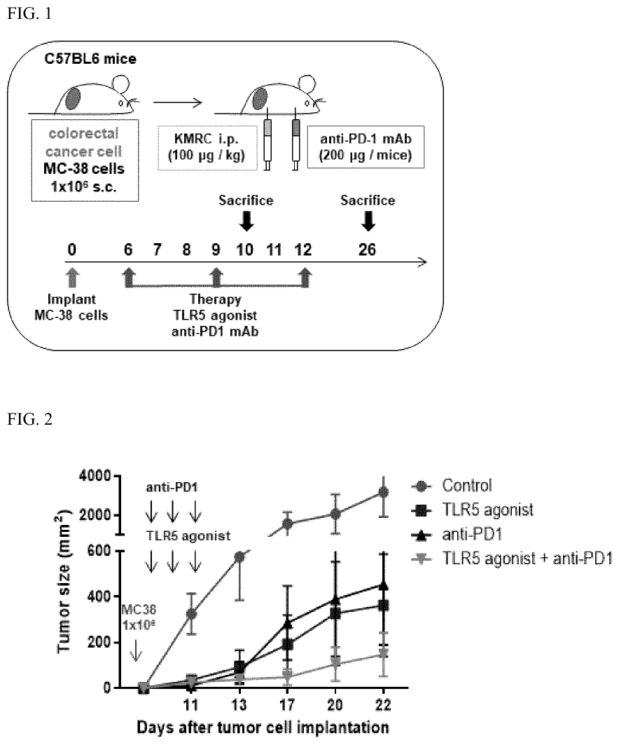

First, after intraperitoneal transplantation of the MC-38 cell line into mouse, every 3 days from the sixth day, 3 times in total, 200 μl/kg of physiological saline (control: •), 100 μg/kg of TLR5 agonist (▪), 200 μg/mice of anti-PD-1 (▴) were administered alone, and anti-PD-1 and TLR5 agonists (▾) were administered concurrently. Then, the anticancer effects were observed, and the results were shown in FIG. 2. As shown in FIG. 2, the TLR5 agonist of the present invention delayed the growth rate of the tumor, and it was confirmed that this anticancer effect was significantly inhibited in tumor growth than in the positive control, anti-PD-1. In addition, through the above experimental results, it can be confirmed that the concurrent administration of the TLR5 agonist of the present invention and the anti-PD-1 has an increased synergistic effect on the antitumor compared to the administration of each substance alone.

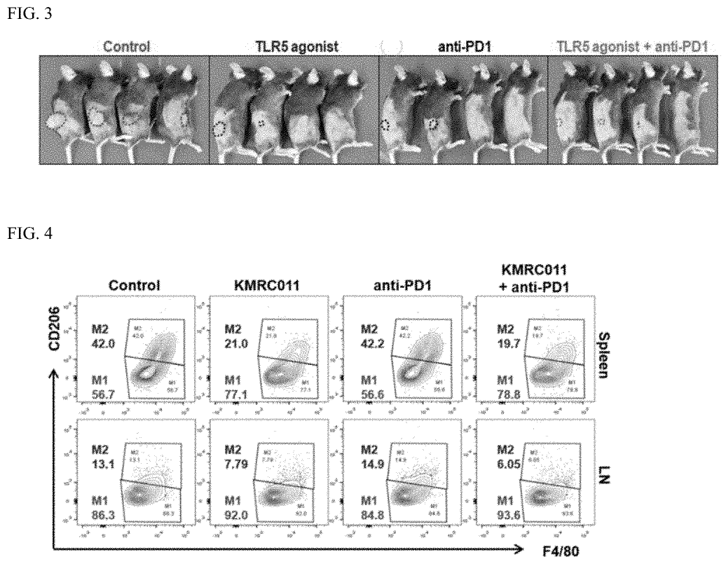

Meanwhile, the mouse transplanted with the MC-38 cell line intraperitoneally were monitored, and the results were shown in FIG. 3. It was observed that the sizes of the cancer in mouse were reduced due to administration of the TLR5 agonist of the present invention when compared to the negative or positive control group. These results show that the TLR5 agonist (“KMRC011”) of the present invention can be used as an effective anticancer agent as it is used as an immune checkpoint blockade, and that it can be used as an anticancer adjuvant having a synergistic effect in tumor treatment when concurrently administered with an existing immune checkpoint blockade.

(2) Confirmation of Tumor Microenvironment Regulation by the TLR5 Agonist

In order to confirm whether the TLR5 agonist of the present invention induces changes in the tumor microenvironment as an immune checkpoint blockade, the immune profiling of mouse spleen and lymph node cells for each administrated substance in a tumor mouse model was evaluated by flow cytometry. To this end, the present inventors established conditions for differentiation of M1 and M2 macrophages from mouse spleen and lymph node cells. In general, M1 macrophages are known to have anticancer effects by showing tumor aggression, and M2 macrophages are known to grow tumors as tumor-supporting macrophages that are friendly to cancer. Therefore, whether the TLR5 agonist of the present invention had an anticancer effect by regulating the tumor microenvironment of the tumor cell line was checked through the change in the degree of polarization of the M1/M2 macrophages.

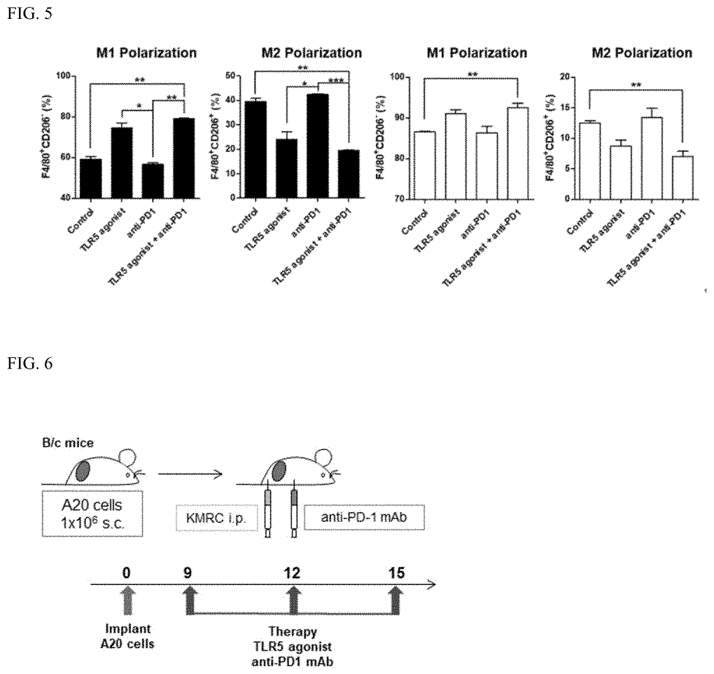

In the tumor animal model, after administration of the saline, the TLR5 agonist, and the anti-PD-1 alone, and the concurrent administration of the anti-PD-1 and the TLR5 agonist, the M1 and M2 macrophages from mouse spleen and lymph node cells were analyzed using flow cytometry. The polarization was checked by observation, and the results were shown in FIGS. 4 and 5. In the case of spleen and lymph node cells treated with the TLR5 agonists, it was confirmed that the polarization of M1 macrophages (F4/80+CD206-) was increased, while the polarization of M2 macrophages (F4/80+CD206+) was decreased. As such, it can be confirmed that the TLR5 agonist can regulate the distribution of immune cell subtypes, M1 and M2 macrophages in order to increase the response of the immune checkpoint blockade.

Therefore, it can be seen that the TLR5 agonist (“KMRC011”) of the present invention can create an environment in which the antitumor effect can be optimally exhibited by changing the tumor microenvironment either alone or concurrently with an existing immune checkpoint blockade.

2. B-Cell Lymphoma Mouse Model

(1) Verification of Bioluminescence of A20-Luc-GFP Cell Line

In the A20-Luc-GFP cell line, the degree of bioluminescence according to the number of cells was checked by measuring the bioluminescence of different cell numbers from 1×10² cells to 1×10⁵ cells, and the results were shown in FIG. 7. It was confirmed that as the number of cells increased, the degree of bioluminescence also increased proportionally. These results mean that when the bioluminescence of the tumor formed when the tumor mouse model was introduced using the A20-Luc-GFP cell line is measured, the degree of tumor growth in vivo can be identified.

(2) Verification of Anticancer Effect by the TLR5 Agonist in the Tumor Mouse Model

In order to observe the anticancer effect of the TLR5 agonist of the present invention (“KMRC011”) administered alone or concurrently, tumor growth was compared for each administered substance after inducing the tumor mouse model.

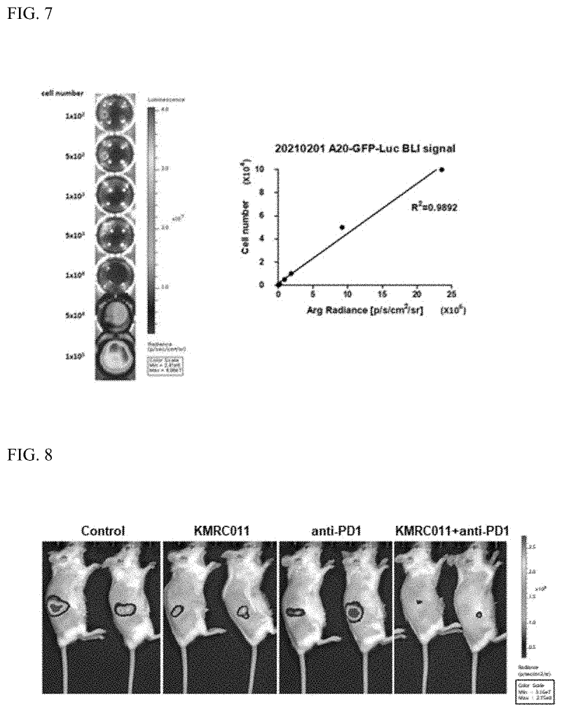

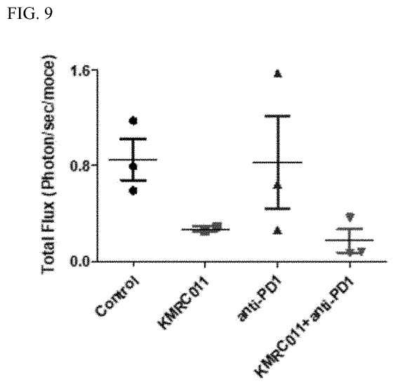

After transplantation of the A20-Luc-GFP cell line into the mouse subcutaneously, 200 μl/mice of physiological saline, 100 μg/kg of the TLR5 agonist, and 200 μg/mice of the anti-PD-1 were administered alone from the ninth day, 3 times in total, at intervals of three days, and the anti-TLR5 agonist and the anti-PD-1 were concurrently administrated. The anticancer effect was observed by measuring bioluminescence, and the results were shown in FIG. 6. As shown in FIGS. 8 to 9, the TLR5 agonist of the present invention retarded the growth rate of tumors. In addition, through the above experimental results, it can be seen that the concurrent administration of the TLR5 agonist of the present invention and the anti-PD-1 has an increased synergistic effect on the antitumor compared to the administration of each substance alone.

The sizes of the tumor formed in the mouse transplanted with the A20-Luc-GFP cell line were measured by BLI, and the results were shown in FIG. 9. When the sizes of the cancers in mouse were compared for the negative control group (•), the TLR5 agonist administration group (▪), and the anti-PD-1 administration group (▴), the decrease in size was observed upon concurrent administration of the TLR5 agonist and the anti-PD-1 of the present invention (▾).

These results confirmed that the TLR5 agonist (“KMRC011”) of the present invention not only exhibited a single antitumor effect, but also could be used as an anticancer adjuvant having a synergistic effect in tumor treatment when concurrently administered with an existing immune checkpoint blockade.

As the specific parts of the present invention have been described in detail above, it is apparent to those of ordinary skill in the art that these specific descriptions are only preferred embodiments, and the scope of the present invention is not limited thereby.

Accordingly, the substantial scope of the present invention will be defined by the appended claims and their equivalents.

### INDUSTRIAL APPLICABILITY

The present invention relates to an anticancer composition including the TLR5 agonist derived from flagellin as an active ingredient. The TLR5 agonist derived from flagellin of the present invention shows an anticancer or anticancer adjuvant effect either alone or concurrently with the immune checkpoint blockade.

What is claimed is:

1. An anticancer composition comprising a therapeutically effective amount of a TLR5 agonist derived from flagellin.

2. The composition according to claim 1, wherein the TLR5 agonist derived from flagellin includes D0 domain and D1 domain of the flagellin.

3. The composition according to claim 1, wherein the TLR5 agonist derived from flagellin includes a linker peptide of an amino acid sequence set forth in SEQ ID NO: 1 inside the D1 domain.

4. The composition according to claim 1, wherein the TLR5 agonist derived from flagellin includes an amino acid sequence set forth in SEQ ID NO: 2.

5. The composition according to claim 1, wherein the TLR5 agonist derived from flagellin regulates tumor microenvironment.

6. The composition according to claim 1, wherein the TLR5 agonist derived from flagellin increases M1 (F4/80+CD206−) polarization and decreases M2 (F4/80+CD206+) polarization in a spleen and lymph node.

7. The composition according to claim 1, wherein the composition has a growth inhibitory activity of a cancer cell.

8. The composition according to claim 1, further comprising an immune checkpoint blockade.

9. The composition according to claim 8, wherein the immune checkpoint blockade is an anti-PD-1 antibody.

10. The composition according to claim 9, wherein the anti-PD-1 antibody is one or more selected from the group consisting of avelumab, tremelimumab, ipilimumab, nivolumab, pembrolizumav, atezolizumab, durvalumab, lamvrolizumab, AMP-224, MEDI4376 and CT-011.

11. The composition according to claim 1, wherein the composition includes a pharmaceutically acceptable carrier.

12. A pharmaceutical agent comprising the composition of claim 11.
